(19) United States
(12) Patent Application Publication
(10) Pub. No.: US 2024/0101648 A1
(43) Pub. Date: Mar. 28, 2024

(54) ANTIBODIES AGAINST RESPIRATORY SYNCYTIAL VIRUS, HUMAN METAPNEUMOVIRUS AND PNEUMONIA VIRUS OF MICE AND METHODS OF USING THE SAME

(71) Applicant: Humabs BioMed SA, Bellinzona (CH)

(72) Inventor: Davide CORTI, Bellinzona (CH)

(21) Appl. No.: 18/264,408

(22) PCT Filed: Feb. 8, 2022
(86) PCT No.: PCT/US2022/015652

§ 371 Date: Aug. 4, 2023

Related U.S. Application Data
(60) Provisional application No. 63/147,676, filed on Feb. 9, 2021.

(51) Int. Cl. C07K 16/10; A61P 31/14; C12N 15/63

(52) U.S. Cl. CPC C07K 16/1027; A61P 31/14; C12N 15/63; C07K 2317/14; C07K 2317/94

(57) ABSTRACT

The instant disclosure provides antibodies that can bind to a paramyxovirus and neutralize an infection by the paramyxovirus. The paramyxovirus can be, for example, respiratory syncytial virus, metapneumovirus, or pneumonia virus of mice. The antibodies comprise modifications in the Fc region that improve in vivo stability of the antibodies, one or more effector function of the antibodies, or both. Antibody compositions, polynucleotides that encode the antibodies, vectors, host cells, and methods of using the antibodies to prevent and/or treat a paramyxovirus infection are also provided.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930485_434WO_SEQUENCE_LISTING.txt. The text file is 29.7 KB, was created on Feb. 7, 2022, and is being submitted electronically via EFS-Web.

### BACKGROUND

Modalities for preventing or treating infection by paramyxoviruses, such as respiratory syncytial virus, metapneumovirus, or pneumonia virus of mice, are needed.

### BRIEF DESCRIPTION OF THE DRAWINGS

FIG. 1 shows the design of a preclinical study evaluating in vivo stability of human IgG1 antibodies RSV_Ab (HC: SEQ ID NO 0.5; LC: SEQ ID NO.:4) and RSV_Ab_MLNS (HC: SEQ ID NO.:1; LC: SEQ ID NO.:4) in cynomolgus monkeys. Stability was determined by correlating total human IgG1 concentration in plasma (using a mouse monoclonal antibody (mAb) specific for human IgG1 CH2) vs. neutralizing activity.

FIG. 2 shows results from the preclinical study depicted in FIG. 1. Pharmacokinetics (PK) was calculated as antibody concentration in plasma as measured by ELISA, captured by coated Ds-Cav1 F antigen.

FIG. 3 shows further results from the preclinical study depicted in FIG. 1. Data show blood PK of RSV_Ab (top) and RSV_Ab_MLNS (bottom). For two of the three animals that received RSV_Ab_MLNS, PK was continued to day 113. Quantification was based on DsCav1-based antigen capture ELISA (animals were pre-screened to be non-immune to RSV): (re)analysis was performed in parallel on all samples. There was no clear anti-drug antibody (ADA) response in any of the 12 animals (3 of the 9 animals received engineered anti-FluA antibodies).

### DETAILED DESCRIPTION

Provided herein are antibodies that can bind to a paramyxovirus and neutralize an infection by a paramyxovirus. The paramyxovirus can be, for example, respiratory syncytial virus, metapneumovirus, or pneumonia virus of mice. The antibodies comprise modifications in the Fc that improve in vivo stability of the antibodies, one or more effector function of the antibodies, or both.

In some embodiments, following a single dose of the antibody to a subject (e.g. administered by slow i.v. infusion), the antibody is present in plasma of the subject at a concentration greater than or equal to the neutralization EC50 (the concentration of antibody required for half-maximal neutralization) of the antibody for up to 100, up to 200, up to 300, up to 400, up to 500, up to 600, up to 700, up to 800, up to 900, up to 1000, up to 1,100, up to 1,200, or up to 1,300 hours, or more. Neutralization EC50 may be informed by results from an in vitro assay and/or by results from an in vivo assay. In some embodiments, an antibody comprises a neutralization EC50 for RSV of about 20 micrograms (such as, for example, 20 micrograms) per ml, optionally in an in vitro neutralization assay and/or in a cynomolgus monkey.

For example, in some embodiments, following a single dose (e.g. administered by slow i.v. infusion) of the antibody at 15 mg/kg to a cynomolgus monkey, the antibody is present in plasma of the subject at a concentration greater than or equal to the neutralization EC50 for up to 100, up to 200, up to 300, up to 400, up to 500, up to 600, up to 700, up to 800, up to 900, up to 1000, up to 1,100, up to 1,200, or up to 1,300 hours, or more.

In some embodiments, following a single dose (e.g. administered by slow i.v. infusion) of the antibody at 15 mg/kg to a cynomolgus monkey, the antibody is present in plasma at a concentration greater than or equal to 20 micrograms per ml for over 1,000 hours, over 1,050 hours, over 1,100 hours, over 1,150 hours, over 1,200 hours, over 1,250 hours, over 1300 hours, or more, or up to about 1,250 hours, or up to 1,300 hours, or up to 1,200 hours, or up to 1,100 hours, or up to 1,000 hours. In certain embodiments, the antibody comprises a neutralization EC50 for RSV of about 20 micrograms (such as, for example, 20 micrograms) per ml, optionally in an in vitro neutralization assay and/or in a cynomolgus monkey.

In some embodiments, following a single dose of the antibody (e.g. administered by slow i.v. infusion) at 15 mg/kg to a cynomolgus monkey, the antibody is present in plasma at a concentration greater than or equal to 20 micrograms per ml for over 10 days, over 20 days, over 30 days, over 40 days, over 50 days, or for up to 55 days, or for up to 50 days, or for up to 60 days. In certain embodiments, the antibody comprises a neutralization EC50 for RSV of about 20 micrograms (such as, for example, 20 micrograms) per ml, optionally in an in vitro neutralization assay and/or in a cynomolgus monkey.

In some embodiments, at 113 days following a single dose of the antibody or antibody composition (e.g. administered by slow i.v. infusion) at 15 mg/kg to a cynomolgus monkey, the antibody is present in plasma at a concentration between 2 micrograms per ml and 10 micrograms per ml, or between 2 micrograms per ml and 7 micrograms per ml, or about 2, about 3, about 4, about 5, about 6, or about 7 micrograms per ml.

In some embodiments, following a single dose of the antibody or antibody composition (e.g. administered by slow i.v. infusion) at 15 mg/kg to a cynomolgus monkey, the antibody is present in plasma in accordance with any one or more of the concentrations at a given time according to Table A.

TABLE A
Antibody Concentration in Cynomolgus Monkey Plasma Over Time
Following a Single Dose of Antibody or Antibody at 15 mg/kg

| | Days |
|---|---|
| | following |
| | the Single |
| | Dose of |
| Antibody Plasma Concentration (micrograms/mL) | Antibody |
| Greater than or about 100, or in a range from about 70 to | 10 |
| about 150, or in a range from about 70 to about 200, or in a | |
| range from about 70 to about 120, or in a range from about | |
| 80 to about 150, or about 70, about 80, about 90, about 100, | |
| about 110, about 120, about 130, about 140, or about 150, | |
| about 160, about 170, about 180, about 190, or about 200 | |
| About 100, or in a range from about 50 to about 100, or | 20 |
| about 50, about 60, about 70, about 80, or about 90 | |
| In a range from about 40 to about 80, or in a range from | 30 |
| about 40 to about 70, or about 40, about 45, about 50, about | |
| 55, about 60, about 65, about 70, or about 75, or about 80 | |
| In a range from about 20 to about 50, or in a range from | 40 |
| about 20 to about 60, or in a range from about 30 to about | |
| 50, or in a range from about 30 to about 60, or about 20, | |
| about 25, about 30, about 35, about 40, about 45, about 50, | |
| about 55, or about 60 | |
| In a range from about 20 to about 40, or in a range from | 50 |
| about 25 to 40, or about 20, about 25, about 30, about 35, or | |
| about 40 | |
| In a range from about 15 to about 30, or in a range from | 60 |
| about 20 to 30, or about 15, about 20, about 25, or about 30 | |
| In a range from about 15 to about 20, or about 15, about 16, | 70 |
| about 17, about 18, about 19, or about 20 | |
| In a range from about 10 to about 19, or in a range from | 80 |
| about 10 to about 18, or in a range from about 11 to about | |
| 18, or in a range from about 11 to about 19, or about 10, | |
| about 11, about 12, about 13, about 14, about 15, about 16, | |
| about 17, about 18, or about 19 | |
| In a range from about 8 to about 11, or in a range from 8 to | 90 |
| 11, or in a range from about 8 to about 12, or in a range from | |
| 8 to 12, or about 8, about 9, about 10, about 11, or about 12 | |
| In a range from about 6 to about 9, or in a range from 6 to 9, | 100 |
| or about 6, about 7, about 8, or about 9 | |
| In a range from about 4 to about 7, or in a range from 4 to 7, | 110 |
| or in a range from about 5 to about 7, or about 4, about 5, | |
| about 6, or about 7 | |

Plasma concentration of an antibody can be quantified using, for example, DsCav1-based antigen capture ELISA. The presence of virus or viral load in a subject (e.g. RSV in a cynomolgus monkey) can be assessed using known techniques and a sample such as, for instance, a nasal swab.

In some embodiments, the antibodies do not elicit an anti-drug antibody (ADA) response in cynomolgus monkeys; for example, as measured at day 56 following administration of a single dose of an antibody or antibody composition at 15 mg/kg by slow i.v. infusion.

Also provided are polynucleotides that encode the antibodies vectors, host cells, and related compositions, as well as methods of using the antibodies, nucleic acids, vectors, host cells, and related compositions to prevent or treat (e.g., reduce, delay, eliminate, or prevent) a paramyxovirus infection in a subject and/or in the manufacture of a medicament for preventing or treating a paramyxovirus infection in a subject.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

In some embodiments, the polynucleotide (e.g. mRNA) comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine. These features are known in the art and are discussed in, for example, Zhang et al. Front. Immunol., DOI=10.3389/fimmu.2019.00594 (2019); Eyler et al. PNAS 116(46): 23068-23071; DOI: 10.1073/pnas.1821754116 (2019); Nance and Meier, ACS Cent. Sci. 2021, 7, 5, 748-756; doi.org/10.1021/acscentsci.1c00197 (2021), and van Hoecke and Roose, J. Translational Med 17:54 (2019); https://doi.org/10.1186/s12967-019-1804-8, which modified nucleosides and mRNA features are incorporated herein by reference.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, polynucleotide, vector, host cell, or composition that is outside of a subject, such as outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Such an organism, microorganism, cell, nucleic acid molecule, or vector can also be described as “modified”. Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon. A polypeptide encoded by an engineered non-natural nucleic acid molecule can be described as an “engineered” polypeptide.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding an antibody (or other polypeptide), or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host (e.g. human) or a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host or a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host or a host cell.

The term “construct” refers to any polynucleotide that contains a recombinant nucleic acid molecule (or, when the context clearly indicates, a protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mot. Ther. 8:108, 2003: Mates et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation.

The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA: sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao el al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure). “Host” can also refer to a subject to whom a nucleic acid encoding an antibody is administered, and/or who has a paramyxovirus such as, for example, respiratory syncytial virus, metapneumovirus, or pneumovirus of mice.

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

“Antigen” or “Ag”, as used herein, refers to an immunogenic molecule that provokes an immune response. This immune response may involve antibody production, activation of certain immunologically-competent cells, activation of complement, antibody dependent cytotoxicicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in a paramyxovirus (e.g., an F protein or portion thereof), such as present in a virion, or expressed or presented on the surface of a cell infected a paramyxovirus.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

Antibodies

In some embodiments, the present disclosure provides an isolated antibody comprising: (i) two heavy chain polypeptides each having (i.e. comprising or consisting of) the amino acid sequence of SEQ ID NO.:1; and (ii) two light chain polypeptides each having (i.e. comprising or consisting of) the amino acid sequence of SEQ ID NO.:4.

In other embodiments, the present disclosure provides an isolated antibody comprising: (i) two heavy chain polypeptides each having (i.e. comprising or consisting of) the amino acid sequence of SEQ ID NO.:2; and (ii) two light chain polypeptides each having (i.e. comprising or consisting of) the amino acid sequence of SEQ ID NO.:4.

In still other embodiments, the present disclosure provides an isolated antibody comprising: (i) two heavy chain polypeptides each having (i.e. comprising or consisting of) the amino acid sequence of SEQ ID NO.:3; and (ii) two light chain polypeptides each having (i.e. comprising or consisting of) the amino acid sequence of SEQ ID NO.:4.

In some embodiments, the present disclosure provides an isolated antibody comprising: (i) two heavy chain polypeptides each comprising the amino acid sequence of SEQ ID NO.:6; and (ii) two light chain polypeptides each comprising the amino acid sequence of SEQ ID NO.:4.

In some embodiments, the present disclosure provides an isolated antibody comprising: (i) two heavy chain polypeptides each comprising the amino acid sequence of SEQ ID NO.:7; and (ii) two light chain polypeptides each comprising the amino acid sequence of SEQ ID NO.:4.

In some embodiments, the present disclosure provides an isolated antibody comprising: (i) two heavy chain polypeptides each comprising the amino acid sequence of SEQ ID NO.:8; and (ii) two light chain polypeptides each comprising the amino acid sequence of SEQ ID NO.:4.

The two heavy chain polypeptides of an antibody of the present disclosure associate to form a homodimer. One of the two heavy chain polypeptides further associates with one of the two light chain polypeptides, and the other of the two heavy chain polypeptides further associates with the other of the two light chain polypeptides.

The antibodies are capable of binding to a paramyxovirus and neutralizing an infection by the paramyxovirus. The paramyxovirus can be, for example, respiratory syncytial virus, metapneumovirus, or pneumonia virus of mice.

As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody is capable of preventing and/or neutralizing a paramyxovirus infection in an in vitro model of infection and/or in an in vivo animal model of infection and/or in a human.

Terms understood by those in the art of antibody technology are each given the meaning acquired in the art, unless expressly defined differently herein. For example, the term “antibody” refers herein to an intact antibody comprising at least two heavy (H) chains (HCs; also called heavy chain polypeptides) and two light (L) chains (LCs; also called light chain polypeptides)) inter-connected by disulfide bonds (i.e. LC—HC-HC-LC, as a bivalent tetramer molecule). The term encompasses intact or full-length antibodies, including antibodies of any class or sub-class, including IgG and sub-classes thereof (IgG1, IgG2, IgG3, IgG4), IgM, IgE, IgA, and IgD.

The terms “V_{L}” or “VL” and “V_{H}” or “VH” refer to the variable binding region from an antibody light chain and an antibody heavy chain, respectively. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs. However, in some cases, a VH alone, a VL alone, or one, two, three, four, or five of the CDRs are involved in antigen-binding.

Numbering of CDR and framework regions may be according to any known method or scheme, such as, for example, the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immwnol. 27:55, 2003; Honegger and Plackthun, J. Mol. Bio. 309:657-670 (2001); and the antibody numbering method developed by the Chemical Computing Group (CCG); e.g., using Molecular Operating Environment (MOE) software (www.chemcomp.com). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH 1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody of the present disclosure comprises a CL, a CH1, a CH2, and a CH3.

A “Fab” (fragment antigen binding) is the part of an antibody that binds to antigens and includes the variable region and CH1 of the heavy chain linked to the light chain via one or more inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although sometimes at a lower affinity than the entire binding site.

Presently disclosed antibodies comprise a Fc polypeptide. An “Fc” dimer comprises the carboxy-terminal portions (i.e., the CH2 and CH3 domains of IgG) of both antibody H chains held together by disulfides. Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

In some embodiments, an antibody comprises a Fc polypeptide comprising mutations selected from (EU numbering with reference to human IgG1): G236A; S239D; A330L; and 1332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/1332E. In some embodiments, the Fc polypeptide or fragment thereof does not comprise S239D. In some embodiments, the Fc polypeptide or fragment thereof comprises S at position 239. GAALIE improves binding affinity to FcRyIIa and FcγRIIIa and reduces binding affinity to FcγRIIb.

In certain embodiments, the Fc polypeptide comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide (e.g., as compared to a reference Fc polypeptide or antibody that is otherwise the same but does not comprise the modification(s)). In certain embodiments, the Fc polypeptide comprises or is derived from a IgG (e.g., IgG1) Fc and a half-life-extending mutation comprises any one or more of: M428L; N434S; N434H; N434A; N434S; M252Y; S254T; T256E; T250Q; P2571 Q311I; D376V; T307A; E380A (EU numbering with reference to human IgG1). In certain embodiments, a half-life-extending mutation comprises M428UN434S (also referred to herein as “MLNS” or “LS”). In certain embodiments, a half-life-extending mutation comprises M252Y/S254T/T256E. In certain embodiments, a half-life-extending mutation comprises T250Q/M428L. In certain embodiments, a half-life-extending mutation comprises P2571/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, an antibody includes a Fc polypeptide that comprises the substitution mutations M428L/N434S. In some embodiments, an antibody includes a Fc polypeptide that comprises the substitution mutations G236A/A330UI332E. In certain embodiments, an antibody includes a (e.g., IgG1) Fc polypeptide that comprises a G236A mutation, an A330L mutation, and a 1332E mutation (GAALIE), and does not comprise a S239D mutation (e.g., comprises a native S at position 239). In particular embodiments, an antibody includes an Fc polypeptide that comprises the substitution mutations: M428L/N434S and G236A/A330UI332E, and optionally does not comprise S239D. In certain embodiments, an antibody includes a Fc polypeptide that comprises the substitution mutations: M428UN434S and G236A/S239D/A330IJ1332E (EU numbering with reference to human IgG1).

It will be understood that, for example, production in a mammalian cell line can remove one or more C-terminal lysine of an antibody heavy chain polypeptide (see, e.g., Liu et al. mAbs 6(5):1145-1154 (2014)). Accordingly, an antibody of the present disclosure can comprise a heavy chain polypeptide wherein a C-terminal lysine residue is present or is absent; in other words, encompassed are embodiments where the C-terminal residue of a heavy chain polypeptide is not a lysine, and embodiments where a lysine is the C-terminal residue. In certain embodiments, a composition comprises a plurality of an antibody of the present disclosure, wherein one or more antibody does not comprise a lysine residue at the C-terminal end of the heavy chain polypeptide, and wherein one or more antibody comprises a lysine residue at the C-terminal end of the heavy chain polypeptide.

Accordingly, in some embodiments, the present disclosure provides an isolated antibody comprising: (i) two heavy chain polypeptides each comprising the amino acid sequence of SEQ ID NO.:6, SEQ ID NO.:7, or SEQ ID NO.:8; and (ii) two light chain polypeptides each comprising the amino acid sequence of SEQ ID NO.4.

In certain embodiments, the antibody comprises a mutation that alters glycosylation, wherein the mutation that alters glycosylation comprises N297A, N297Q, or N297G, and/or the antibody is partially or fully aglycosylated and/or is partially or fully afucosylated. Host cell lines and methods of making partially or fully aglycosylated or partially or fully afucosylated antibodies are known (see, e.g., PCT Publication No. WO 2016/181357; Suzuki et al. Clin. Cancer Res. 13(6).1875-82 (2007); Huang et al. MAbs 6:1-12 (2018)). Fucosylation of an antibody can be effected by introducing amino acid mutations to introduce or disrupt a fucosylation site; by expressing the antibody in a host cell which has been genetically engineered to lack the ability (or have an inhibited or compromised ability) to fucosylate the antibody; by expressing the antibody under conditions in which a host cell is impaired in its ability to fucosylate the antibody (e.g., in the presence of 2-fluoro-L-fucose (2FF)), or the like.

In certain embodiments, an antibody: is afucosylated; has been produced in a host cell that is incapable of fucosylation or that is inhibited in its ability to fucosylate the antibody; has been produced under conditions that inhibit fucosylation thereof by a host cell; or any combination thereof.

In certain embodiments, the antibody is capable of eliciting continued protection in vivo in a subject even once no detectable levels of the antibody can be found in the subject (i.e., when the antibody has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody comprises one or more modifications, such as, for example, mutations in the Fc comprising G236A, A330L, and 1332E, that are capable of activating dendritic cells that may induce, e.g., T cell immunity to the antigen.

In any of the presently disclosed embodiments, the antibody can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

A “human antibody” is an antibody containing only sequences that are present in an antibody that is produced by a human. However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

Polynucleotides, Vectors, and Host cells

In another aspect, the present disclosure provides isolated polynucleotides that encode any of the presently disclosed antibodies or a portion thereof (e.g., a CDR, a VH, a VL, a heavy chain, or a light chain). Disclosed polynucleotides include, for example, those that encode a heavy chain of an antibody, a light chain of an antibody, a heavy chain and a light chain of an antibody, or two heavy chains and two light chains of an antibody.

In certain embodiments, the polynucleotide is codon-optimized for expression in a host cell. From a known a coding sequence, codon optimization can be performed using known techniques and tools, e.g., using the GenScript@ OptimiumGene tool or Gene Synthesis by GeneArt® (ThermoFisher); see also Scholten el al., Clinl. Immunol. 119:135, 2006). Codon-optimized sequences include sequences that are partially codon-optimized (i.e., one or a plurality of codons is optimized for expression in the host cell) and those that are fully codon-optimized.

It will also be appreciated that polynucleotides encoding antibodies of the present disclosure may possess different nucleotide sequences while still encoding a same antibody due to, for example, the degeneracy of the genetic code, splicing, and the like.

It will be appreciated that in certain embodiments, a polynucleotide encoding an antibody or portion thereof is comprised in a polynucleotide that includes other sequences and/or features for, e.g., expression of the antibody or portion thereof in a host cell. Exemplary features include a promoter sequence, a polyadenylation sequence, a sequence that encodes a signal peptide (e.g., located at the N-terminus of an expressed antibody heavy chain or light chain), or the like.

In any of the presently disclosed embodiments, the polynucleotide can comprise deoxyribonucleic acid (DNA) or ribonucleic acid (RNA). In some embodiments, the RNA comprises messenger RNA (mRNA).

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine.

Vectors are also provided, wherein the vectors comprise or contain a polynucleotide as disclosed herein. A vector can comprise any one or more of the vectors disclosed herein. In particular embodiments, a vector is provided that comprises a DNA plasmid construct encoding the antibody or a portion thereof (e.g., so-called “DMAb”; see, e.g., Muthumani et al., Jinfect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody are encoded by a polynucleotide comprised in a single plasmid. In other embodiments, the substituent components of the antibody are encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase). In some embodiments, a vector of the present disclosure comprises a nucleotide sequence encoding a signal peptide. The signal peptide may or may not be present (e.g., can be enzymatically cleaved from) on the mature antibody. In some embodiments, a vector of the present disclosure comprises a polyadenylation signal sequence.

In some embodiments, a vector of the present disclosure comprises a CMV promoter.

In some embodiments, a method is provided that comprises administering to a subject a first polynucleotide (e.g., mRNA) encoding an antibody heavy chain, a VH, or a Fd (VH+CH1), and administering to the subject a second polynucleotide (e.g., mRNA) encoding the cognate antibody light chain, VL, or VL+CL.

In some embodiments, a polynucleotide (e.g., mRNA) is provided that encodes a heavy chain and a light chain of an antibody. In some embodiments, a polynucleotide (e.g., mRNA) is provided that encodes two heavy chains and two light chains of an antibody. See, e.g. Li, JQ., Zhang, ZR., Zhang, H Q. et al. Intranasal delivery of replicating mRNA encoding neutralizing antibody against SARS-CoV-2 infection in mice. Sig Transduct Target Ther 6, 369 (2021). https://doi.org/10.1038/s41392-021-00783-1, the antibody-encoding mRNA constructs, vectors, and related techniques of which are incorporated herein by reference. In some embodiments, a polynucleotide is delivered to a subject via an alphavirus replicon particle (VRP) delivery system. In some embodiments, a replicon comprises a modified VEEV replicon comprising two subgenomic promoters. In some embodiments, a polynucleotide or replicon can translate simultaneously the heavy chain (or VH, or VH+1) and the light chain (or VL, or VL+CL) of an antibody. In some embodiments, a method is provided that comprises delivering to a subject such a polynucleotide or replicon.

In a further aspect, the present disclosure also provides a host cell expressing an antibody according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E coli. The expression of peptides in prokaryotic cells such as E co/i is well established (see, e.g., Pluckthun, A. Bio-Technology 9:545-551 (1991). For example, antibodies may be produced in bacteria, in particular when glycosylation and Fc effector function are not needed. For expression of antibody polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing an antibody according to the present disclosure. In such embodiments, the cells may be stably transfected with the vector as described herein. Alternatively, cells may be transiently transfected with a vector according to the present disclosure encoding an antibody as disclosed herein. In any of the presently disclosed embodiments, a polynucleotide may be heterologous to the host cell.

Accordingly, the present disclosure also provides recombinant host cells that heterologously express an antibody the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody in nature. Moreover, the host cell may impart a post-translational modification (PTM; e.g., glycosylation or fucosylation) on the antibody that is not present in a native state of the antibody (or in a native state of a parent antibody from which the antibody was engineered or derived). Such a PTM may result in a functional difference (e.g., reduced immunogenicity). Accordingly, an antibody of the present disclosure that is produced by a host cell as disclosed herein may include one or more post-translational modification that is distinct from the antibody (or parent antibody) in its native state (e.g., a human antibody produced by a CHO cell can comprise a more post-translational modification that is distinct from the antibody when isolated from the human and/or produced by the native human B cell or plasma cell).

Insect cells useful expressing a binding protein of the present disclosure are known in the art and include, for example, Spodopterafrugipera Sf¹9 cells, Trichoplusia ni BTI-TN5B1-4 cells, and Spodopterafrugipera SfSWTO1 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodopterafrugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing an antibody of the present disclosure. For example, PLANTIBODIES^{T}M technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a Y0 cell, a Sp2/0 cell, a NS0 cell, a human liver cell, a myeloma cell, or a hybridoma cell.

In a related aspect, the present disclosure provides methods for producing an antibody, wherein the methods comprise culturing a host cell of the present disclosure under conditions and for a time sufficient to produce the antibody. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

Compositions

Also provided herein are compositions that comprise any one or more of the presently disclosed antibodies, polynucleotides, vectors, or host cells, singly or in any combination, and can further comprise a pharmaceutically acceptable carrier, excipient, or diluent. Carriers, excipients, and diluents are discussed in further detail herein.

In certain embodiments, a composition comprises a plurality of an antibody of the present disclosure, wherein one or more antibody of the plurality does not comprise a lysine residue at the C-terminal end of the heavy chain polypeptide, and wherein one or more antibody of the plurality comprises a lysine residue at the C-terminal end of the heavy chain polypeptide.

In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of the antibody. In certain embodiments, a composition comprises a polynucleotide (e.g., mRNA) coupled to a suitable delivery vehicle or carrier.

Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 1J(2):e1530 (2019)). Principles, reagents, and techniques for designing appropriate mRNA and formulating mRNA-LNP and delivering the same are described in, for example, Pardi el al. (J Control Re/ease 217345-351 (2015)); Thess et al. (Mol Ther 23: 1456-1464 (2015)); Thran et al. (EMBO)MolMed 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

Methods and Uses

Also provided herein are methods for use of an antibody, nucleic acid, vector, cell, or composition of the present disclosure in the diagnosis of a paramyxovirus (e.g., in a human subject, or in a sample obtained from a human subject).

Methods of diagnosis (e.g., in vitro, ex vivo) may include contacting an antibody with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody with a sample. Such a detection step can be performed at the bench, i.e. without any contact to the human or animal body. Examples of detection methods are well-known to the person skilled in the art and include, e.g., ELISA (enzyme-linked immunosorbent assay), including direct, indirect, and sandwich ELISA.

Also provided herein are methods of treating a subject using an antibody of the present disclosure, or a composition comprising the same, or a nucleic acid, vector, or host cell of the present disclosure, or a composition comprising the same, wherein the subject has, is believed to have, or is at risk for having an infection by a paramyxovirus, such as, for example, respiratory syncytial virus, metapneumovirus, or pneumonia virus of mice. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody, nucleic acid, vector, host cell, or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a paramyxovirus infection (i.e., in a statistically significant manner). In certain embodiments, therapeutic or prophylactic/preventive benefit includes a reduced duration of hospitalization for treatment of a paramyxovirus infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of an antibody, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

Accordingly, in certain embodiments, methods are provided for treating a paramyxovirus (e.g., RSV, MPV, or PVM) infection in a subject, wherein the methods comprise administering to the subject an effective amount of an antibody, polynucleotide, vector, host cell, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

In certain embodiments, a human subject treated according to the present disclosure is an infant, a child, an adolescent, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 0-21 years old, 20-44 years old, 45-54 years old, 55-64 years old, 65-74 years old, 75-84 years old, or 85 years old, or older. In some embodiments, a subject is a pediatric subject. Pediatric subjects include persons aged 21 or younger at the time of diagnosis or treatment. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, treatment is administered as peri-exposure prophylaxis.

Typical routes of administering the presently disclosed compositions thus include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, polynucleotide, vector, host cell, or composition to the subject.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody as herein disclosed such that a suitable dosage will be obtained. Typically, this amount is at least 0.01% of the antibody in the composition. When intended for oral administration, this amount may be varied to be between 0.1 and about 70% of the weight of the composition. Certain oral pharmaceutical compositions contain between about 4% and about 75% of the antibody. In certain embodiments, pharmaceutical compositions and preparations according to the present invention are prepared so that a parenteral dosage unit contains between 0.01 to 10% by weight of antibody prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody in the aqueous delivery system.

In general, an appropriate dose and treatment regimen provide the composition(s) in an amount sufficient to provide therapeutic and/or prophylactic benefit (such as described herein, including an improved clinical outcome (e.g., a longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat a paramyxovirus infection), which will vary depending upon a variety of factors including the activity of the specific compound employed: the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective daily dose of an antibody is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody.

In certain embodiments, a method comprises administering the antibody, polynucleotide, vector, host cell, or composition to the subject at 2, 3, 4, 5, 6, 7, 8, 9, 10 times, or more.

In certain embodiments, a method comprises administering the antibody, polynucleotide, vector, host cell, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, polynucleotide, vector, host cell, or composition at least one time prior to the subject being infected by a paramyxovirus.

Compositions comprising an antibody, polynucleotide, vector, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising an antibody of the disclosure and each active agent in its own separate dosage formulation. For example, an antibody as described herein and the other active agent can be administered to the patient together in a single oral dosage composition such as a tablet or capsule, or each agent administered in separate oral dosage formulations. Similarly, an antibody as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody and one or more additional active agents can be administered at essentially the same time, i.e., concurrently, or at separately staggered times, i.e., sequentially and in any order; combination therapy is understood to include all these regimens.

In certain embodiments, a combination therapy is provided that comprises one or more antibody (or one or more nucleic acid, host cell, vector, or composition) of the present disclosure and one or more anti-inflammatory agent and/or one or more anti-viral agent. In particular embodiments, the one or more anti-inflammatory agent comprises a corticosteroid such as, for example, dexamethasone, prednisone, or the like. In some embodiments, the one or more anti-inflammatory agents comprise a cytokine antagonist such as, for example, an antibody that binds to IL6 (such as siltuximab), or to IL-6R (such as tocilizumab), or to IL-1β, IL-7, IL-8, IL-9, IL-10, FGF, G-CSF, GM-CSF, IFN-γ, IP-10, MCP-1, MIP-1A, MIP1-B, PDGR, TNF-α, or VEGF. In some embodiments, anti-inflammatory agents such as ruxolitinib and/or anakinra are used. In some embodiments, the one or more anti-viral agents comprise nucleotide analogs or nucelotide analog prodrugs such as, for example, remdesivir, sofosbuvir, acyclovir, and zidovudine. In particular embodiments, an anti-viral agent comprises lopinavir, oseltamivir, peramivir, zanamivir, ritonavir, favipiravir, or any combination thereof. In some embodimens, a combination therapy comprises leronlimab. Anti-inflammatory agents for use in a combination therapy of the present disclosure also include non-steroidal anti-inflammatory drugs (NSAIDS). It will be appreciated that in such a combination therapy, the one or more antibody (or one or more nucleic acid, host cell, vector, or composition) and the one or more anti-inflammatory agent and/or one or the more antiviral agent can be administered in any order and any sequence, or together.

In some embodiments, an antibody (or one or more nucleic acid, host cell, vector, or composition) is administered to a subject who has previously received one or more anti-inflammatory agent and/or one or more antiviral agent. In some embodiments, one or more anti-inflammatory agent and/or one or more antiviral agent is administered to a subject who has previously received an antibody (or one or more nucleic acid, host cell, vector, or composition).

In a related aspect, uses of the presently disclosed antibodies, vectors, host cells, and compositions are provided.

In certain embodiments, an antibody, polynucleotide, vector, host cell, or composition is provided for use in a method of treating a paramyxovirus infection in a subject.

In certain embodiments, an antibody, polynucleotide, vector, host cell, or composition is provided for use in a method of manufacturing or preparing a medicament for treating a paramyxovirus infection in a subject.

TABLE 1
Sequences

| | SEQ ID | |
|---|---|---|
| Sequence Description | NO. | Sequence |
| HC of RSV_Ab_MLNS | 1 | EVQLVESGGGLVKPGGSLRLSCA |
| | | ASGFTFSSYSMNWVRQAPGKGLE |
| | | WVSSISASSSYSDYADSAKGRFTIS |
| | | RDNAKTSLFLQMNSLRAEDTAIYF |
| | | CARARATGYSSITPYFDIWGQGTL |
| | | VTVSSASTKGPSVFPLAPSSKSTSG |
| | | GTAALGCLVKDYFPEPVTVSWNS |
| | | GALTSGVHTFPAVLQSSGLYSLSS |
| | | VVTVPSSSLGTQTYICNVNHKPSN |
| | | TKVDKRVEPKSCDKTHTCPPCPAP |
| | | ELLGGPSVFLFPPKPKDTLMISRTP |
| | | EVTCVVVDVSHEDPEVKFNWYV |
| | | DGVEVHNAKTKPREEQYNSTYRV |
| | | VSVLTVLHQDWLNGKEYKCKVS |
| | | NKALPAPIEKTISKAKGQPREPQV |
| | | YTLPPSREEMTKNQVSLTCLVKGF |
| | | YPSDIAVEWESNGQPENNYKTTPP |
| | | VLDSDGSFFLYSKLTVDKSRWQQ |
| | | GNVFSCSVLHEALHSHYTQKSLSL |
| | | SPGK |
| HC of RSV_Ab_GAALIE | 2 | EVQLVESGGGLVKPGGSLRLSCA |
| | | ASGFTFSSYSMNWVRQAPGKGLE |
| | | WVSSISASSSYSDYADSAKGRFTIS |
| | | RDNAKTSLFLQMNSLRAEDTAIYF |
| | | CARARATGYSSITPYFDIWGQGTL |
| | | VTVSSASTKGPSVFPLAPSSKSTSG |
| | | GTAALGCLVKDYFPEPVTVSWNS |
| | | GALTSGVHTFPAVLQSSGLYSLSS |
| | | VVTVPSSSLGTQTYICNVNHKPSN |
| | | TKVDKRVEPKSCDKTHTCPPCPAP |
| | | ELLAGPSVFLFPPKPKDTLMISRTP |
| | | EVTCVVVDVSHEDPEVKFNWYV |
| | | DGVEVHNAKTKPREEQYNSTYRV |
| | | VSVLTVLHQDWLNGKEYKCKVS |
| | | NKALPLPEEKTISKAKGQPREPQV |
| | | YTLPPSREEMTKNQVSLTCLVKGF |
| | | YPSDIAVEWESNGQPENNYKTTPP |
| | | VLDSDGSFFLYSKLTVDKSRWQQ |
| | | GNVFSCSVMHEALHNHYTQKSLS |
| | | LSPGK |
| HC of RSV_Ab_MLNS_GAALIE | 3 | EVQLVESGGGLVKPGGSLRLSCA |
| | | ASGFTFSSYSMNWVRQAPGKGLE |
| | | WVSSISASSSYSDYADSAKGRFTIS |
| | | RDNAKTSLFLQMNSLRAEDTAIYF |
| | | CARARATGYSSITPYFDIWGQGTL |
| | | VTVSSASTKGPSVFPLAPSSKSTSG |
| | | GTAALGCLVKDYFPEPVTVSWNS |
| | | GALTSGVHTFPAVLQSSGLYSLSS |
| | | VVTVPSSSLGTQTYICNVNHKPSN |
| | | TKVDKRVEPKSCDKTHTCPPCPAP |
| | | ELLAGPSVFLFPPKPKDTLMISRTP |
| | | EVTCVVVDVSHEDPEVKFNWYV |
| | | DGVEVHNAKTKPREEQYNSTYRV |
| | | VSVLTVLHQDWLNGKEYKCKVS |
| | | NKALPLPEEKTISKAKGQPREPQV |
| | | YTLPPSREEMTKNQVSLTCLVKGF |
| | | YPSDIAVEWESNGQPENNYKTTPP |
| | | VLDSDGSFFLYSKLTVDKSRWQQ |
| | | GNVFSCSVLHEALHSHYTQKSLSL |
| | | SPGK |
| LC of RSV_Ab, | 4 | QSVVTQPPSVSGAPGQRVTISCTG |
| RSV_Ab_MLNS, | | SSSNIGAGYDVHWYQQLPGTAPK |
| RSV_Ab_GAALIE, and | | LLIYDNNNRPSGVPDRFSASKSGT |
| RSV_Ab MLNS_GAALIE | | SASLAITGLQAEDEADYYCQSYD |
| | | RSLSGVFGTGTKVTVLGQPKAAP |
| | | SVTLFPPSSEELQANKATLVCLISD |
| | | FYPGAVTVAWKADSSPVKAGVET |
| | | TTPSKQSNNKYAASSYLSLTPEQ |
| | | WKSHRSYSCQVTHEGSTVEKTVA |
| | | PTECS |
| HC of RSV_Ab | 5 | EVQLVESGGGLVKPGGSLRLSCA |
| | | ASGFTFSSYSMNWVRQAPGKGLE |
| | | WVSSISASSSYSDYADSAKGRFTIS |
| | | RDNAKTSLFLQMNSLRAEDTAIYF |
| | | CARARATGYSSITPYFDIWGQGTL |
| | | VTVSSASTKGPSVFPLAPSSKSTSG |
| | | GTAALGCLVKDYFPEPVTVSWNS |
| | | GALTSGVHTFPAVLQSSGLYSLSS |
| | | VVTVPSSSLGTQTYICNVNHKPSN |
| | | TKVDKRVEPKSCDKTHTCPPCPAP |
| | | ELLGGPSVFLFPPKPKDTLMISRTP |
| | | EVTCVVVDVSHEDPEVKFNWYV |
| | | DGVEVHNAKTKPREEQYNSTYRV |
| | | VSVLTVLHQDWLNGKEYKCKVS |
| | | NKALPAPIEKTISKAKGQPREPQV |
| | | YTLPPSREEMTKNQVSLTCLVKGF |
| | | YPSDIAVEWESNGQPENNYKTTPP |
| | | VLDSDGSFFLYSKLTVDKSRWQQ |
| | | GNVFSCSVMHEALHNHYTQKSLS |
| | | LSPGK |
| HC of RSV_Ab_MLNS | 6 | EVQLVESGGGLVKPGGSLRLSCA |
| without C-terminal lysine | | ASGFTFSSYSMNWVRQAPGKGLE |
| | | WVSSISASSSYSDYADSAKGRFTIS |
| | | RDNAKTSLFLQMNSLRAEDTAIYF |
| | | CARARATGYSSITPYFDIWGQGTL |
| | | VTVSSASTKGPSVFPLAPSSKSTSG |
| | | GTAALGCLVKDYFPEPVTVSWNS |
| | | GALTSGVHTFPAVLQSSGLYSLSS |
| | | VVTVPSSSLGTQTYICNVNHKPSN |
| | | TKVDKRVEPKSCDKTHTCPPCPAP |
| | | ELLGGPSVFLFPPKPKDTLMISRTP |
| | | EVTCVVVDVSHEDPEVKFNWYV |
| | | DGVEVHNAKTKPREEQYNSTYRV |
| | | VSVLTVLHQDWLNGKEYKCKVS |
| | | NKALPAPIEKTISKAKGQPREPQV |
| | | YTLPPSREEMTKNQVSLTCLVKGF |
| | | YPSDIAVEWESNGQPENNYKTTPP |
| | | VLDSDGSFFLYSKLTVDKSRWQQ |
| | | GNVFSCSVLHEALHSHYTQKSLSL |
| | | SPG |
| HC of RSV_ Ab_GAALIE | 7 | EVQLVESGGGLVKPGGSLRLSCA |
| without C-terminal lysine | | ASGFTFSSYSMNWVRQAPGKGLE |
| | | WVSSISASSSYSDYADSAKGRFTIS |
| | | RDNAKTSLFLQMNSLRAEDTAIYF |
| | | CARARATGYSSITPYFDIWGQGTL |
| | | VTVSSASTKGPSVFPLAPSSKSTSG |
| | | GTAALGCLVKDYFPEPVTVSWNS |
| | | GALTSGVHTFPAVLQSSGLYSLSS |
| | | VVTVPSSSLGTQTYICNVNHKPSN |
| | | TKVDKRVEPKSCDKTHTCPPCPAP |
| | | ELLAGPSVFLFPPKPKDTLMISRTP |
| | | EVTCVVVDVSHEDPEVKFNWYV |
| | | DGVEVHNAKTKPREEQYNSTYRV |
| | | VSVLTVLHQDWLNGKEYKCKVS |
| | | NKALPLPEEKTISKAKGQPREPQV |
| | | YTLPPSREEMTKNQVSLTCLVKGF |
| | | YPSDIAVEWESNGQPENNYKTTPP |
| | | VLDSDGSFFLYSKLTVDKSRWQQ |
| | | GNVFSCSVMHEALHNHYTQKSLS |
| | | LSPG |
| HC of RSV_Ab_MLNS_GAALIE | 8 | EVQLVESGGGLVKPGGSLRLSCA |
| without C-terminal | | ASGFTFSSYSMNWVRQAPGKGLE |
| lysine | | WVSSISASSSYSDYADSAKGRFTIS |
| | | RDNAKTSLFLQMNSLRAEDTAIYF |
| | | CARARATGYSSITPYFDIWGQGTL |
| | | VTVSSASTKGPSVFPLAPSSKSTSG |
| | | GTAALGCLVKDYFPEPVTVSWNS |
| | | GALTSGVHTFPAVLQSSGLYSLSS |
| | | VVTVPSSSLGTQTYICNVNHKPSN |
| | | TKVDKRVEPKSCDKTHTCPPCPAP |
| | | ELLAGPSVFLFPPKPKDTLMISRTP |
| | | EVTCVVVDVSHEDPEVKFNWYV |
| | | DGVEVHNAKTKPREEQYNSTYRV |
| | | VSVLTVLHQDWLNGKEYKCKVS |
| | | NKALPLPEEKTISKAKGQPREPQV |
| | | YTLPPSREEMTKNQVSLTCLVKGF |
| | | YPSDIAVEWESNGQPENNYKTTPP |
| | | VLDSDGSFFLYSKLTVDKSRWQQ |
| | | GNVFSCSVLHEALHSHYTQKSLSL |
| | | SPG |

The present disclosure also provides the following non-limiting enumerated Embodiments.

Embodiment 1. An antibody comprising: (i) two heavy chain polypeptides each having the amino acid sequence of SEQ ID NO.:1; and (ii) two light chain polypeptides each having the amino acid sequence of SEQ ID NO.:4.

Embodiment 2. An antibody comprising: (i) two heavy chain polypeptides each having the amino acid sequence of SEQ ID NO.:2; and (ii) two light chain polypeptides each having the amino acid sequence of SEQ ID NO.:4.

Embodiment 3. An antibody comprising: (i) two heavy chain polypeptides each having the amino acid sequence of SEQ ID NO.:3; and (ii) two light chain polypeptides each having the amino acid sequence of SEQ ID NO.:4.

Embodiment 4. An antibody comprising: (i) two heavy chain polypeptides each comprising the amino acid sequence of SEQ ID NO.:6; and (ii) two light chain polypeptides each comprising the amino acid sequence of SEQ ID NO.:4.

Embodiment 5. An antibody comprising: (i) two heavy chain polypeptides each comprising the amino acid sequence of SEQ ID NO.:7; and (ii) two light chain polypeptides each comprising the amino acid sequence of SEQ ID NO.:4.

Embodiment 6. An antibody comprising: (i) two heavy chain polypeptides each comprising the amino acid sequence of SEQ ID NO.:8; and (ii) two light chain polypeptides each comprising the amino acid sequence of SEQ ID NO.:4.

Embodiment 7. An isolated polynucleotide encoding: (i) the antibody of any one of Embodiments 1-6; (ii) a heavy chain of the antibody of any one of Embodiments 1-6; (iii) a light chain of the antibody of any one of Embodiments 1-6; or (iv) a heavy chain of the antibody of any one of Embodiments 1-6 and a light chain of the antibody of any one of Embodiments 1-6.

Embodiment 8. The polynucleotide of Embodiment 7, wherein the polynucleotide comprises DNA or RNA, wherein, optionally, the RNA comprises mRNA, wherein, further optionally, the mRNA comprises a modified nucleoside, a cap-1 structure, a cap-2 structure, or any combination thereof and/or comprises a pseudouridine, a N6-methyladenonsine, a 5-methylcytidine, a 2-thiouridine, or any combination thereof.

Embodiment 9. A vector comprising the polynucleotide of Embodiment 7 or 8.

Embodiment 10. A host cell comprising the polynucleotide of Embodiment 7 or 8 or the vector of Embodiment 9.

Embodiment 11. A composition comprising the antibody of any one of Embodiments 1-6, the polynucleotide of Embodiment 7 or 8, the vector of Embodiment 9, and/or the host cell of Embodiment 10, and a pharmaceutically acceptable carrier, excipient, or diluent.

Embodiment 12. A method of preventing a paramyxovirus infection in a subject, comprising administering to the subject an effective amount of: the antibody of any one of Embodiments 1-6; the polynucleotide of Embodiment 7 or 8; the vector of Embodiment 9; the host cell of Embodiment 10; and/or the composition of Embodiment 11.

Embodiment 13. A method of treating a paramyxovirus infection in a subject, comprising administering to the subject an effective amount of: the antibody of any one of Embodiments 1-6; the polynucleotide of Embodiment 7 or 8; the vector of Embodiment 9; the host cell of Embodiment 10; and/or the composition of Embodiment 11.

Embodiment 14. The method of Embodiment 12 or 13, wherein the paramyxovirus comprises respiratory syncytial virus, metapneumovirus, pneumoniavirus of mice, or any combination thereof.

Embodiment 15. The antibody of any one of Embodiments 1-6, the polynucleotide of Embodiment 7 or 8, the vector of Embodiment 9, the host cell of Embodiment 10, and/or the composition of Embodiment 11, for use in a method of preventing paramyxovirus infection in a subject, wherein, optionally, the paramyxovirus comprises respiratory syncytial virus, metapneumovirus, pneumoniavirus of mice, or any combination thereof.

Embodiment 16. The antibody of any one of Embodiments 1-6, the polynucleotide of Embodiment 7 or 8, the vector of Embodiment 9, the host cell of Embodiment 10, and/or the composition of Embodiment 11, for use in a method of treating paramyxovirus infection in a subject, wherein, optionally, the paramyxovirus comprises respiratory syncytial virus, metapneumovirus, pneumoniavirus of mice, or any combination thereof.

Embodiment 17. The antibody of any one of Embodiments 1-6, the polynucleotide of Embodiment 7 or 8, the vector of Embodiment 9, the host cell of Embodiment 10, and/or the composition of Embodiment 11, for use in the manufacture of a medicament for preventing a paramyxovirus infection in a subject, wherein, optionally, the paramyxovirus comprises respiratory syncytial virus, metapneumovirus, pneumoniavirus of mice, or any combination thereof.

Embodiment 18. The antibody of any one of Embodiments 1-6, the polynucleotide of Embodiment 7 or 8, the vector of Embodiment 9, the host cell of Embodiment 10, and/or the composition of Embodiment 11, for use in the manufacture of a medicament for treating a paramyxovirus infection in a subject, wherein, optionally, the paramyxovirus comprises respiratory syncytial virus, metapneumovirus, pneumoniavirus of mice, or any combination thereof.

Embodiment 19. A method of making the antibody of any one of Embodiments 1-6, the method comprising culturing a host cell expressing the antibody under conditions and for a time sufficient to produce the antibody.

Embodiment 20. The method of Embodiment 19, further comprising isolating and/or purifying the antibody.

### EXAMPLES

### Example 1

### Pharmacokinetics Study

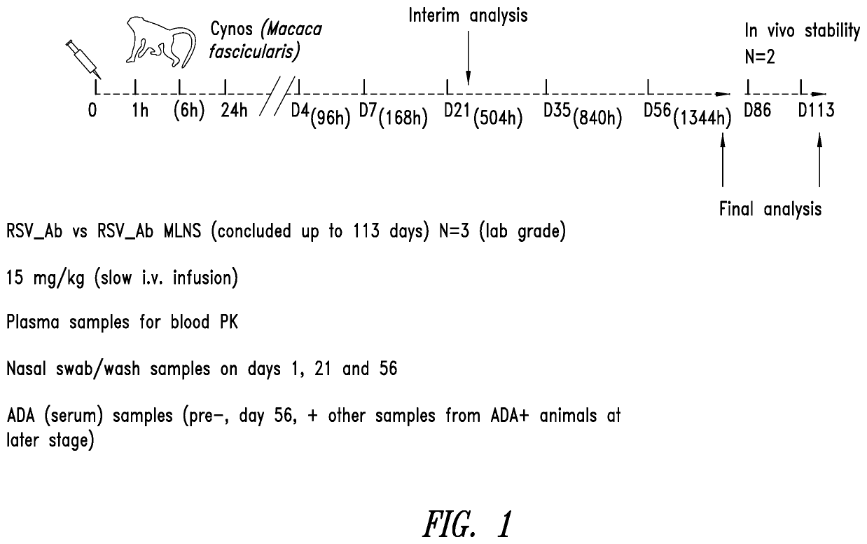

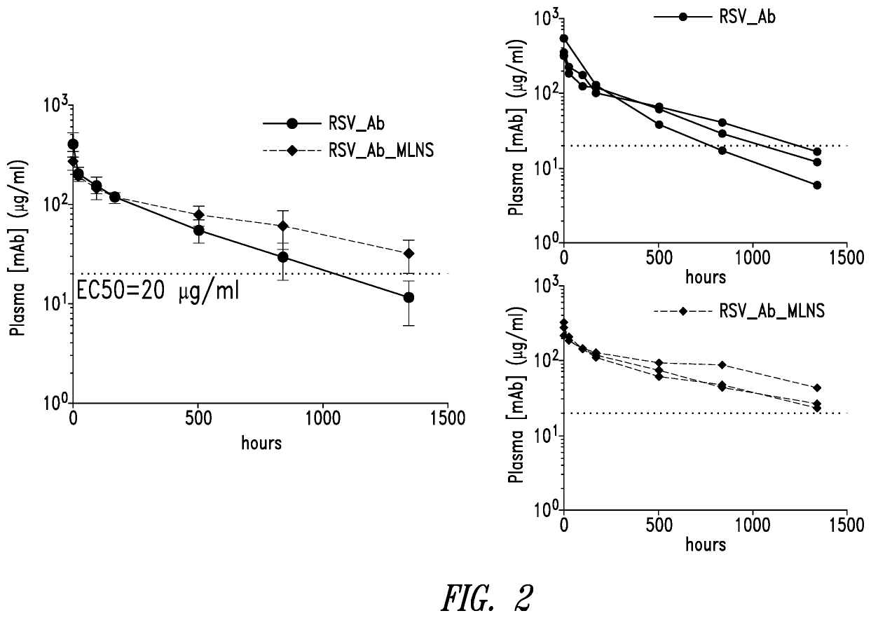

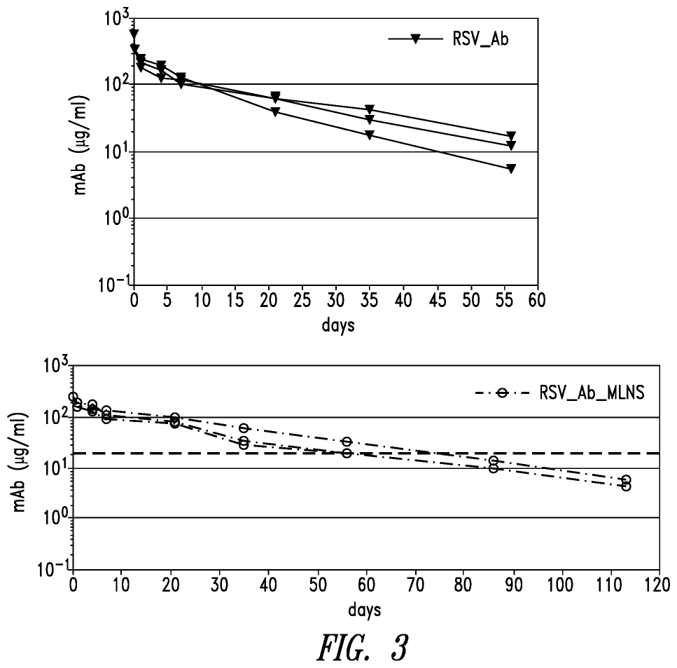

RSV_Ab neutralizes RSV, MPV, and PVM in vitro and in vivo in mice. An in vivo study was performed using cynomolgus monkeys to evaluate the stability of RSV_Ab (IgG1 with HC: SEQ ID NO.:5; LC: SEQ ID NO.:4) and RSV_Ab_MLNS (IgG1 with HC: SEQ ID NO.:1; LC: SEQ ID NO.:4). The study design is shown in FIG. 1. Data are shown in FIGS. 2 and 3. As shown in FIG. 2, following a dose of 15 mg/kg (slow i.v. infusion) RSV_Ab_MLNS, RSV_Ab_MLNS was present in plasma at concentrations exceeding 20 micrograms per ml past 1000 hours post-dose, including past 1250 hours post-dose. By comparison, RSV_Ab was present at lower concentrations. As shown in FIG. 3, RSV_Ab_MLNS had better stability over a longer period of time as compared to RSV_Ab.

### Example 2

### Prophylactic and Therapeutic Use of Rsv_Ab Mlns

RSV_Ab_MLNS, RSV_Ab_GAALIE (IgG1 with HC: SEQ ID NO.:2; LC: SEQ ID NO.:4), or RSV_Ab_MLNS_GAALIE (IgG1 with HC: SEQ ID NO.:3; LC: SEQ ID NO.:4) is administered prophylactically to human subjects at risk for contracting RSV, and therapeutically to human subjects with RSV.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/147,676, filed on Feb. 9, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An antibody comprising:
(i) two heavy chain polypeptides each having the amino acid sequence of SEQ ID NO.:1; and
(ii) two light chain polypeptides each having the amino acid sequence of SEQ ID NO.:4.

2. An antibody comprising:
(i) two heavy chain polypeptides each having the amino acid sequence of SEQ ID NO.:2; and
(ii) two light chain polypeptides each having the amino acid sequence of SEQ ID NO.:4.

3. An antibody comprising:
(i) two heavy chain polypeptides each having the amino acid sequence of SEQ ID NO.:3; and
(ii) two light chain polypeptides each having the amino acid sequence of SEQ ID NO.:4.

4. An antibody comprising:
(i) two heavy chain polypeptides each comprising the amino acid sequence of SEQ ID NO.:6; and
(ii) two light chain polypeptides each comprising the amino acid sequence of SEQ ID NO.:4.

5. An antibody comprising:
(i) two heavy chain polypeptides each comprising the amino acid sequence of SEQ ID NO.:7; and
(ii) two light chain polypeptides each comprising the amino acid sequence of SEQ ID NO.:4.

6. An antibody comprising:
(i) two heavy chain polypeptides each comprising the amino acid sequence of SEQ ID NO.:8; and
(ii) two light chain polypeptides each comprising the amino acid sequence of SEQ ID NO.:4.

7. An isolated polynucleotide encoding:
(i) the antibody of any one of claims 1-6;
(ii) a heavy chain of the antibody of any one of claims 1-6;
(iii) a light chain of the antibody of any one of claims 1-6; or
(iv) a heavy chain of the antibody of any one of claims 1-6 and a light chain of the antibody of any one of claims 1-6.

8. The polynucleotide of claim 7, wherein the polynucleotide comprises DNA or RNA, wherein, optionally, the RNA comprises mRNA, wherein, further optionally, the mRNA comprises a modified nucleoside, a cap-1 structure, a cap-2 structure, or any combination thereof and/or comprises a pseudouridine, a N6-methyladenonsine, a 5-methylcytidine, a 2-thiouridine, or any combination thereof.

9. A vector comprising the polynucleotide of claim 7 or 8.

10. A host cell comprising the polynucleotide of claim 7 or 8 or the vector of claim 9.

11. A composition comprising the antibody of any one of claims 1-6, the polynucleotide of claim 7 or 8, the vector of claim 9, and/or the host cell of claim 10, and a pharmaceutically acceptable carrier, excipient, or diluent.

12. A method of preventing a paramyxovirus infection in a subject,
comprising administering to the subject an effective amount of: the antibody of any one of claims 1-6; the polynucleotide of claim 7 or 8; the vector of claim 9; the host cell of claim 10; and/or the composition of claim 11.

13. A method of treating a paramyxovirus infection in a subject, comprising administering to the subject an effective amount of: the antibody of any one of claims 1-6; the polynucleotide of claim 7 or 8; the vector of claim 9; the host cell of claim 10; and/or the composition of claim 11.

14. The method of claim 12 or 13, wherein the paramyxovirus comprises respiratory syncytial virus, metapneumovirus, pneumoniavirus of mice, or any combination thereof.

15. The antibody of any one of claims 1-6, the polynucleotide of claim 7 or 8, the vector of claim 9, the host cell of claim 10, and/or the composition of claim 11, for use in a method of preventing paramyxovirus infection in a subject, wherein, optionally, the paramyxovirus comprises respiratory syncytial virus, metapneumovirus, pneumoniavirus of mice, or any combination thereof.

16. The antibody of any one of claims 1-6, the polynucleotide of claim 7 or 8, the vector of claim 9, the host cell of claim 10, and/or the composition of claim 11, for use in a method of treating paramyxovirus infection in a subject, wherein, optionally, the paramyxovirus comprises respiratory syncytial virus, metapneumovirus, pneumoniavirus of mice, or any combination thereof.

17. The antibody of any one of claims 1-6, the polynucleotide of claim 7 or 8, the vector of claim 9, the host cell of claim 10, and/or the composition of claim 11, for use in the manufacture of a medicament for preventing a paramyxovirus infection in a subject, wherein, optionally, the paramyxovirus comprises respiratory syncytial virus, metapneumovirus, pneumoniavirus of mice, or any combination thereof.

18. The antibody of any one of claims 1-6, the polynucleotide of claim 7 or 8, the vector of claim 9, the host cell of claim 10, and/or the composition of claim 11, for use in the manufacture of a medicament for treating a paramyxovirus infection in a subject, wherein, optionally, the paramyxovirus comprises respiratory syncytial virus, metapneumovirus, pneumoniavirus of mice, or any combination thereof.

19. A method of making the antibody of any one of claims 1-6, the method comprising culturing a host cell expressing the antibody under conditions and for a time sufficient to produce the antibody.

20. The method of claim 19, further comprising isolating and/or purifying the antibody.


SEQUENCE LISTING (8 sequences)

SEQ ID NO: 1 - protein, 454 aa, Artificial sequence
  Synthetic sequence HC of RSV_Ab_MLNS
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 2 - protein, 454 aa, Artificial sequence
  Synthetic sequence HC of RSV_Ab_ GAALIE
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 454 aa, Artificial sequence
  Synthetic sequence HC of RSV_Ab_ MLNS_GAALIE
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 4 - protein, 216 aa, Artificial sequence
  Synthetic sequence LC of RSV_Ab, RSV_Ab_MLNS, RSV_Ab_GAALIE, and RSV_Ab_MLNS_GAALIE
QSVVTQPPSVSGAPGQRVTISCTGSSSNIGAGYDVHWYQQLPGTAPKLLIYDNNNRPSGVPDRFSASKSGTSASLAITGLQAEDEADYYCQSYDRSLSGVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 454 aa, Artificial sequence
  Synthetic sequence HC of RSV_Ab
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 453 aa, Artificial sequence
  Synthetic sequence HC of RSV_Ab_MLNS without C-terminal lysine
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 7 - protein, 453 aa, Artificial sequence
  Synthetic sequence HC of RSV_Ab_ GAALIE without C-terminal lysine
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 453 aa, Artificial sequence
  Synthetic sequence HC of RSV_Ab_ MLNS_GAALIE without C-terminal lysine
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG